(19) United States
(12) Patent Application Publication
(10) Pub. No.: US 2008/0131532 A1
(43) Pub. Date: Jun. 5, 2008

(54) Fast asleep

(76) Inventors: Lorn Leitman, Miami, FL (US); Gustavo Barni, Pompano Beach, FL (US)

Correspondence Address: Lorn Leitman, 8660 West Flagler St., #200, Miami, FL, 33144, US

(21) Appl. No.: 11/900,582

(22) Filed: Sep. 12, 2007

Related U.S. Application Data
(63) Continuation-in-part of application No. 11/581,558, filed on Oct. 17, 2006, now abandoned.

(51) Int. Cl. A61K 36/67; A61P 25/00

(52) U.S. Cl. 424/734

(57) ABSTRACT

Fast Asleep is an innovative new product with the latest and most effective delivery system. Fast Asleep comes in a dissolving strip very similar to Listerine®.

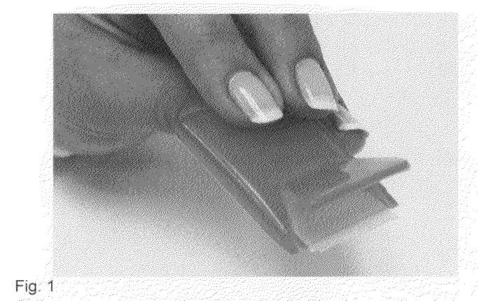

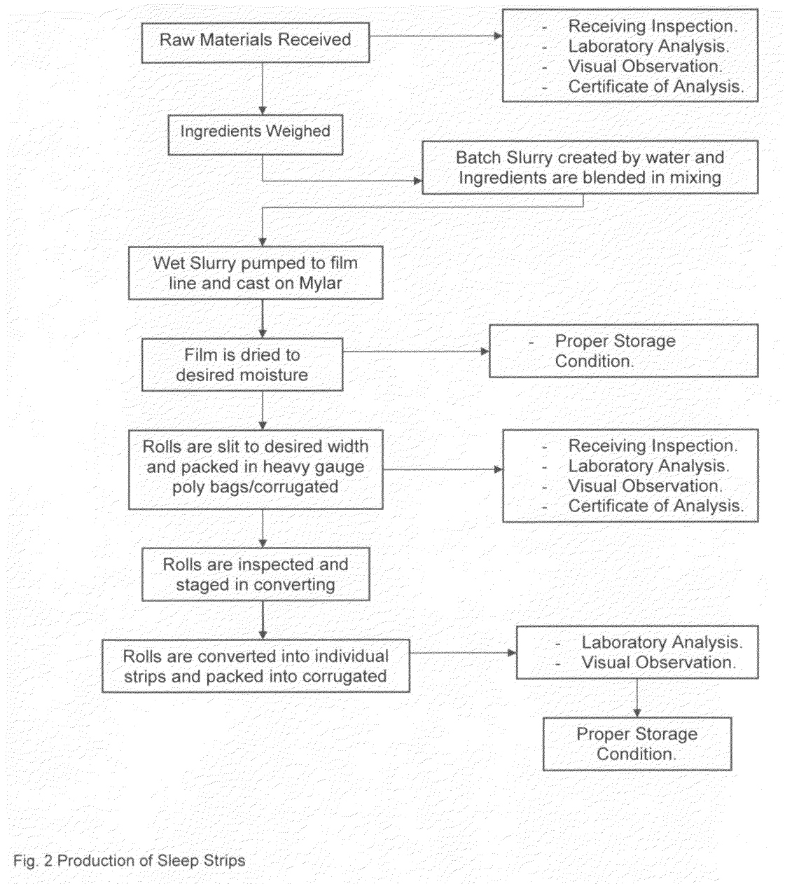

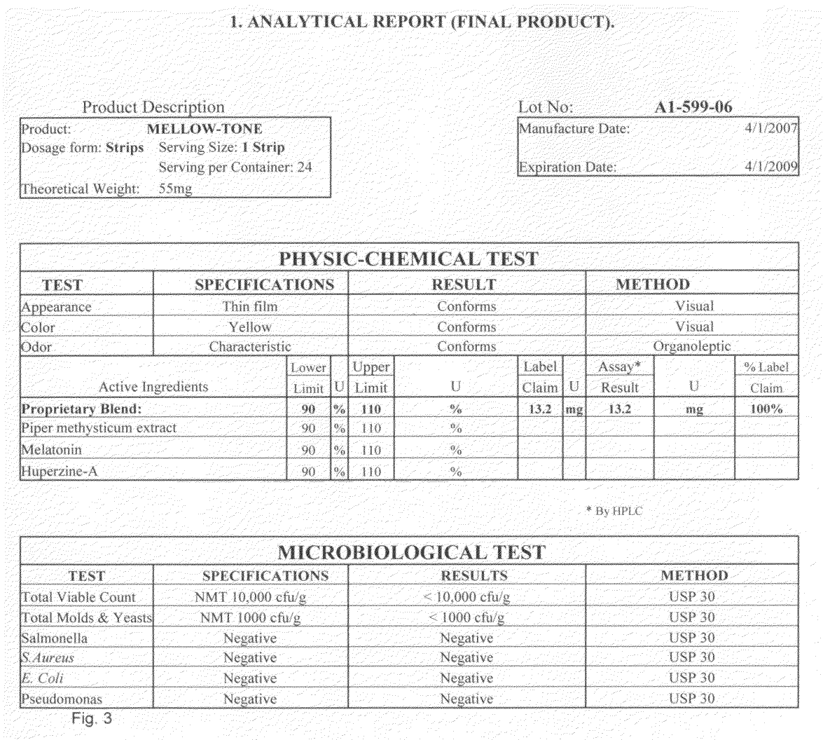

An herbal composition for inducing sleep while simultaneously enhancing memory, Fast Asleep's oral strips contain a unique blend which provides hormone, natural herb and plant alkaloid. The blend consists of Kava-Kava extract (standardized to 30% kavalactones), Melatonin and Huperzine-A. The hormone and the sleep-inducing natural herb are in a range of 5% to 10% by weight of the composition and the memory-enhancing plant alkaloid is approximately 0.1% by weight of the composition.

### CROSS REFERENCE TO RELATED APPLICATIONS

Application is a Continuation in Part of application Ser. No. 11/581,558 filed on Oct. 17, 2006.

### BACKGROUND

Most adults need at least eight hours of sleep every night to be well rested. Not everyone gets the sleep they need. Millions of Americans suffer from sleep problems every year.

Fast Asleep is a natural supplement, delivered in a strip form that induces sleep, facilitates relaxation and enhances memory. Fast Asleep is a combination of three powerful ingredients: Kava-Kava extract standardized to 30% kavalactones, Melatonin and Huperzine-A. Fast Asleep's blend of these three active ingredients has been shown to alleviate and/or prevent:
Insomnia: Difficulty falling asleep and difficulty staying asleep
Anxiety, stress, and depression
Poor concentration and focus; difficulty with memory

Insomnia and other sleep related problems are treated today in several ways; prescription drugs, which can be addictive, over-the counter products and natural products which are sold in tablet form. This method of delivery takes much longer in order to be effective due to the time it takes for the product to be digested and aborbed by the body.

### DESCRIPTION OF PRIOR ART

The patents listed below have been classified for treatment, Sleep Disorders/Circadian Rhythms, none of them have been created in film or strip form.

Patents for Treatment, Sleep Disorders/Circadian Rhythms
1. Autonomic nerve regulating agent
2. Sleep inducing toothpaste made with natural herbs and a natural hormone
3. Screening and therapeutic methods for promoting wakefulness and sleep
4. Delivery of alprazolam, estazolam, midazolam or triazolam through an inhalation route
5. Method of treating sleeplessness with melatonin on an acute basis
6. Methods and compositions for improving sleep
7. Neuropharmacological treatments of sleep-related breathing disorders
8. Tryptophan source from plants and uses therefor
9. Administering bacteria to improve sleep
10. Mediation of circadian rhythms
11. Methods and compositions for treating or preventing sleep disturbances and associated illnesses using very low doses of cyclobenzaprine
12. Use of serotonin agonists to alleviate disordered breathing episodes in a mammal
13. Method for treating or preventing sleep disorders
14. Pharmacological treatment for sleep apnea
15. Sleep quality improvement using a growth hormone secretagogue

1. Autonomic Nerve Regulating Agent
Title: Autonomic nerve regulating agent
U.S. Pat. No. 7,125,911
Issued: Oct. 24, 2006
Inventors: Nagashima; Yoshinao (Tokyo, JP), Sugata; Keiichi (Tokyo, JP), Yada; Yukihiro (Tokyo, JP), Fukuda; Kazuyuki (Tokyo, JP)
Assignee: Kao Corporation (Tokyo, JP)
application Ser. No. 09/972,887

Filed: Oct. 10, 2001

Abstract

The autonomic nerve regulating agent of the present invention, which, has sedative action, sleep inducing action, and stress mitigating action in individuals, regardless of individual variation in sensitivity to or preference for fragrance, contains as an active ingredient a sesquiterpene alcohol with a boiling point of 250.degree. C. or higher, particularly cedrol.

2. Sleep Inducing Toothpaste Made with Natural Herbs and a Natural Hormone
Title: Sleep inducing toothpaste made with natural herbs and a natural hormone
U.S. Pat. No. 6,998,112
Issued: Feb. 14, 2006
Inventors: Zuckerman; Arthur (614 Second Ave., Suite D, New York, N.Y. 10016)
Appl. No.: 391004
Filed: Mar. 18, 2003

Abstract

A toothpaste composition for inducing sleep while simultaneously promoting intraoral cleanliness, which includes toothpaste base ingredients and at least one sleep-inducing natural herb or hormone. The sleep-inducing natural herbs and hormone are selected from the group consisting of Chamomile, Lemon Balm, Passion Flower, and Valerian, and the hormone Melatonin.

3. Screening and Therapeutic Methods for Promoting Wakefulness and Sleep
Title: Screening and therapeutic methods for promoting wakefulness and sleep
U.S. Pat. No. 6,884,596
Issued: Apr. 26, 2005
Inventors: Civelli; Olivier (Irvine, Calif.); Lin; Steven (Upland, Calif.)
Assignee: The Regents of the University of California (Oakland, Calif.)
Appl. No.: 932161
Filed: Aug. 17, 2001

Abstract

The invention provides methods of screening for a compound for promoting wakefulness in a mammal. The method is practiced by providing a compound that is a PrRP receptor agonist and determining the ability of the compound to promote wakefulness. Also provided by the invention are methods of screening for a compound for promoting sleep in a mammal. The methods are practiced by providing a compound that is a PrRP receptor antagonist and determining the ability of the compound to promote sleep. In addition, the invention provides a method of promoting wakefulness in a mammal. The method is practiced by administering to a mammal an effective amount of a PrRP receptor agonist. The invention further provides a method of promoting sleep in a mammal. The method is practiced by administering to a mammal an effective amount of a PrRP receptor antagonist.

4. Delivery of Alprazolam, Estazolam, Midazolam or Triazolam Through an Inhalation Route
Title: Delivery of alprazolam, estazolam, midazolam or triazolam through an inhalation route
U.S. Pat. No. 6,737,043
Issued: May 18, 2004
Inventors: Rabinowitz; Joshua D. (Mountain View, Calif.); Zaffaroni; Alejandro C. (Atherton, Ca)
Assignee: Alexza Molecula Delivery Corporation (Palo Alto, Calif.)
Appl. No.: 155373
Filed: May 22, 2002

Abstract

The present invention relates to the delivery of alprazolam, estazolam, midazolam or triazolam through an inhalation route. Specifically, it relates to aerosols containing alprazolam, estazolam, midazolam or triazolam that are used in inhalation therapy. In a composition aspect of the present invention, the aerosol comprises particles comprising at least 5 percent by weight of alprazolam, estazolam, midazolam or triazolam. In a method aspect of the present invention, alprazolam, estazolam, midazolam or triazolam is delivered to a mammal through an inhalation route. The method comprises: a) heating a composition, wherein the composition comprises at least 5 percent by weight of alprazolam, estazolam, midazolam or triazolam, to form a vapor; and, b) allowing the vapor to cool, thereby forming a condensation aerosol comprising particles, which is inhaled by the mammal. In a kit aspect of the present invention, a kit for delivering alprazolam, estazolam, midazolam or triazolam through an inhalation route to a mammal is provided which comprises: a) a composition comprising at least 5 percent by weight of alprazolam, estazolam, midazolam or triazolam; and, b) a device that forms an alprazolam, estazolam, midazolam or triazolam containing aerosol from the composition, for inhalation by the mammal.

5. Method of Treating Sleeplessness with Melatonin on an Acute Basis
Title: Method of treating sleeplessness with melatonin on an acute basis
U.S. Pat. No. 6,703,412
Issued: Mar. 9, 2004
Inventors: Rosenthal; Holly A. (11 Pine Glen, Blauvelt, N.Y. 10913)
Appl. No.: 255255

Filed: Sep. 27, 2002

A method of treating sleeplessness in a human comprising administering to said human suffering from said sleeplessness an effective sleep-inducing amount of not greater than about 5 mg of melatonin or a pharmaceutically acceptable salt thereof, said administration being at a point in time after said human attempts to go to sleep until no less than one hour prior to said patient's desired awakening time.

6. Methods and Compositions for Improving Sleep
Title: Methods and compositions for improving sleep
U.S. Pat. No. 6,586,478
Issued: Jul. 1, 2003
Inventors: Ackman; C. Bruce (Kingston, Calif.); Adams; Michael A. (Kingston, Calif.); Heaton; Jeremy P. W. (Gananoque, Calif.); Ratz; Jodan D. (Kingston, Calif.)
Assignee: Cellegy Canada (Kingston, Calif.)
Appl. No.: 791127
Filed: Feb. 22, 2001

Abstract

Methods and compositions for improving sleep in individuals with sleep disorders or other conditions which interfere with normal sleep via administration of a NO-mimetic are provided.

7. Neuropharmacological Treatments of Sleep-Related Breathing Disorders
Title: Neuropharmacological treatments of sleep-related breathing disorders
U.S. Pat. No. 6,555,564
Issued: Apr. 29, 2003
Inventors: Radulovacki; Miodrag (Chicago, Ill.); Carley; David W. (Evanston, Ill.)
Assignee: The Board of Trustees of the University of Illinois (Urbana, Ill.)
Appl. No.: 914900
Filed: Nov. 6, 2001
PCT Filed: Mar. 3, 2000
PCT NO: PCT/US00/05834
PCT PUB. NO.: WO00/51590
PCT PUB. Date: Sep. 8, 2000

Abstract

The present invention relates generally to pharmacological methods for the prevention or amelioration of sleep-related breathing disorders via administration of agents or combinations of agents that possess glutamate-related and/or glycine-related pharmacological activity or that modulate the release of either glutamate or glycine (or both) from nerve terminals with the central nervous system.

8. Tryptophan Source from Plants and Uses Therefor
Title: Tryptophan source from plants and uses therefor
U.S. Pat. No. 6,503,543
Issued: Jan. 7, 2003
Inventors: Hudson; Craig J. (253 Cambria Street, Strafford, Ontario, Calif. N5A 1H9); Hudson; Susan P. (253 Cambria Street, Strafford, Ontario, Calif. N5A 1H9)
Appl. No: 580914
Filed: May 26, 2000

Abstract

Compositions are described comprising at least partially defatted meal from a plant source naturally containing tryptophan, preferably squash seeds, and a carbohydrate source provided in an amount capable of facilitating transport of the tryptophan across the blood brain barrier. Also described are dietary supplements, foods and beverages comprising the composition of the invention to induce sleep or provide tryptophan supplementation to individuals in need thereof.

9. Administering Bacteria to Improve Sleep
Title: Administering bacteria to improve sleep
U.S. Pat. No. 6,444,203
Issued: Sep. 3, 2002
Inventors: Krueger; James M. (Pullman, Wash.); Pabst; Michael J. (Germantown, Tenn.); Cayuela; Chantal (Paris, FR); Degivry; Marie-Christine (Le Plessis-Robinson, FR); Hartley; Donna (Arlington, Tex.)
Assignee: Compagnie Gervais Danone (Paris, FR)
Appl. No.: 466768
Filed: Dec. 20, 1999

Abstract

A method of improving sleep in a mammal having a sleep disorder is disclosed. The method includes identifying the mammal having a sleep disorder and then administering Lactobacillus acidophilus CNCM I-2274, Lactobacillus acidophilus CNCM I-2132, Lactobacillus helveticus CNCM I-2275, Streptococcus thermophilus CNCM I-1520, Streptococcus thermophilus CNCM I-2272 or mixtures thereof. The method increases the length of the non rapid eye movement sleep phase and decreases the length of the rapid eye movement sleep phase. The bacteria can be administered in an orally consumable food product or a dietary supplement.

10. Mediation of Circadian Rhythms
Title: Mediation of circadian rhythms
U.S. Pat. No. 6,403,651
Inventors: Kennaway; David (South Australia, AU)
Assignee: Luminis Pty Limited (South Australia, AU)
Appl. No.: 402024
Filed: Mar. 6, 2000
PCT Filed: Mar. 26, 1998
PCT NO: PCT/AU98/00207
371 Date: Mar. 6, 2000
102(e) Date: Mar. 6, 2000
PCT PUB. NO.: WO98/42331
PCT PUB. Date: Oct. 1, 1998
Foreign Application Priority Data: Mar. 26, 1997-[AU] (P0 5882)

Abstract

Method for mediating the effects of light on melatonin rhythmicity in mammals and a method of mediating circadian rhythms, effected by the administration of a compound or compounds effective at a 5-HT2c serotonin receptor site. By administration of selected doses of the 5-HT2c receptor active compound it is possible to advance or delay circadian rhythms as measured by the rate of melatonin production or moderation of core body temperature rhythms.

11. Methods and Compositions for Treating or Preventing Sleep Disturbances and Associated Illnesses Using Very Low Doses of Cyclobenzaprine
Title: Methods and compositions for treating or preventing sleep disturbances and associated illnesses using very low doses of cyclobenzaprine
U.S. Pat. No. 6,395,788
Inventors: Iglehart, I I I; Iredell W. (Baltimore, Md.)
Assignee: Vela Pharmaceuticals, Inc. (Lawrenceville, N.J.)
Appl. No.: 637557
Filed: Aug. 11, 2000

Abstract

The present invention relates to methods and compositions comprising a very low dose of cyclobenzaprine or metabolite thereof for preventing and treating sleep disturbances and illnesses manifested with sleep dysfunction including fibromyalgia syndrome, chronic fatigue syndrome, sleep disorders, psychogenic pain disorders or chronic pain syndromes or symptoms thereof. The present invention further relates to methods and compositions for treating sleep disturbances, chronic pain or fatigue in humans suffering from fibromyalgia syndrome, chronic fatigue syndrome, sleep disorders, psychogenic pain disorders, chronic pain syndromes using a very low dose of cyclobenzaprine.

12. Use of Serotonin Agonists to Alleviate Disordered Breathing Episodes in a Mammal.
Title: Use of serotonin agonists to alleviate disordered breathing episodes in a mammal
U.S. Pat. No. 6,387,907
Inventors: Hendricks; Joan C. (Fort Washington, Pa.); Kubin; Leszek (Havertown, Pa.); Pack; Allan I. (Glen Mills, Pa.); Veasey; Sigrid C. (Philadelphia, Pa.)
Assignee: The Trustees of the University of Pennsylvania (Philadelphia, Pa.)
Appl. No.: 439799

Filed: Nov. 12, 1999

Abstract

The invention includes compositions for alleviating or preventing a disordered breathing episode. The composition of the invention comprises a serotonin re-uptake inhibitor, a TRH agonist and an agent selected from the group consisting of a serotonin precursor and a serotonin agonist.

13. Method For Treating or Preventing Sleep Disorders
Title: Method for treating or preventing sleep disorders
U.S. Pat. No. 6,348,485
Inventors: Ohkawa; Shigenori (Takatsuki, JP); Miyamoto; Masaomi (Takarazuka, JP)
Assignee: Takeda Chemical Industries, Ltd. (Osaka, JP)
Appl. No.: 700405
Filed: Nov. 14, 2000
PCT Filed: Jun. 8, 1999
PCT NO: PCT/JP99/03057
371 Date: Nov. 14, 2000
102(e) Date: Nov. 14, 2000
PCT PUB. NO. WO99/63977
PCT PUB. Date: Dec. 16, 1999
Foreign Application Priority Data: Jun. 9, 1998-[JP] (10-160270)

Abstract

The present invention provides a pharmaceutical composition for treating or preventing sleep disorders which comprises (S)—N-[2-(1,6,7,8-tetrahydro-2H-indeno[5,4-b]furan-8-yl)ethyl]propionamide in combination with at least one active component selected from zolpidem, zopiclone, triazolam and brotizolam.

14. Pharmacological Treatment for Sleep Apnea
Title: Pharmacological treatment for sleep apnea
U.S. Pat. No. 6,331,536
Inventors: Radulovacki; Miodrag (Chicago, Ill.); Carley; David W. (Evanston, Ill.)
Assignee: The Board of Trustees of the University of Illinois (Urbana, Ill.)
Appl. No.: 622823
Filed: Aug. 23, 2000
PCT Filed Feb. 26, 1999
PCT NO: PCT/US99/04347
371 Date: Aug. 23, 2000
102(e) Date: Aug. 23, 2000
PCT PUB. NO.: WO99/43319
PCT PUB. Date: Sep. 2, 1999

Abstract

The present invention relates generally to pharmacological methods for the prevention of amelioration of sleep-related breathing disorders via administration of agents or combinations of agents that possess serotonin-related pharmacological activity.

15. Sleep Quality Improvement Using a Growth Hormone Secretagogue
Title: Sleep quality improvement using a growth hormone secretagogue
U.S. Pat. No. 6,313,133
Inventors: Van Cauter; Eve (Chicago, Ill.); Copinschi; Georges (Brussels, BE)
Assignee: Arch Development Corporation (Chicago, Ill.)
Appl. No.: 492852
Filed: Jan. 27, 2000

Abstract

Methods for re-establishing normal sleep patterns in adults with age-related sleep disorders are provided. In particular, methods are disclosed wherein a compound that stimulates growth hormone and/or prolactin secretion is orally administered to subjects just prior to retiring.

### BRIEF SUMMARY OF INVENTION

Fast Asleep is unique from prior art in that it is a blend of Melatonin, Kava Kava extract and Huperzine-A not found in any other product and is administered by means of a fast dissolving strip which is absorbed through the baccal mucosa. Administration of one strip is believed to elevate the melatonin level due to the location of absorption within minutes (most trial participants felt tired and drowsy within 10-15 minutes), and maintain its effect for at least eight hours.

Fast Asleep Strips help promote:
Increased sleep
Proper function of the pineal gland
Restful, restorative sleep
Relief of jet lag
Strengthening of the immune system
Improvement in overall mood
Relaxation, stress and anxiety relief
Enhanced mental activity
Sharper focus and concentration
Proper memory function

### DETAILED DESCRIPTION OF INVENTION

Test Method of Melatonin

Determination of Melatonin by HPLC

To define a procedure for the quantitative determination of Melatonin (N-[2-(5-Methoxy-1H-indol-3-yl)]ethyl acetamide) using reverse phase high pressure chromatography with UV detection (222 nm).

Apparatus:
High performance liquid chromatograph
Analytical Balance capable of measuring 0.1 mg
0.45 mcm, 47 mm filters
0.2 mcm, 13 mm Nylon Acrodisc membrane filters
C18, 150 mm×4.6 mm analytical column, Prodigy 5 mcm ODS (3) 100 A.
C18, 30 mm×4.6 mm 5 mcm ODS 3, 100 A guard column
General laboratory equipment
Column oven with a regulator to maintain temperature at 35° C.

Reagents:
Water, HPLC grade
Methanol, HPLC grade
Acetonitrile HPLC grade
Phosphoric Acid ACS grade, 88.7% pure
Melatonin standard (Sigma Chemical)

Mobile Phase Preparation:
Add 6.25 mL Phosphoric Acid to a 1000 mL of HPLC water and mix (Solution A)
ACN (Solution B)
Each solution must be individually filtered thru a 0.45 mcm filter and degassed appropriately.
Mix isocratically at the pump 70% A:30% B

Standard Preparation:

Melatonin
Weigh 15.0 mg of melatonin and dissolve in 5 mL of Methanol. Dilute to 50 mL with Solution A.
Melatonin 0.30 mg/mL
The standard can be kept refrigerated and is stable for a several days.

Mix Standard Preparation:

Take 1 mL of each of the above standards and dilute to 10 mL with Solution A Melatonin 0.030 mg/mL

Sample Preparation and Sample Treatment:
Grind ten strips with a mortar and transfer an accurate weight to a 125 mL erlenmeyer flask.
Dissolve a sample containing approximate 3 mg of melatonin/325 mg sample in 10 ml of Methanol, and 90 mL of Solution A.
Sonicate for 10 min, and then stir for 15 minutes.
Filter the extract through a 0.2 mcm membrane filter into amber vials.

Procedure:
Equilibrate the HPLC system for no less than 30 min, and run 1 standard to verify the retention.
Do a System Suitability with 5 injections of the same standard to obtain an RSD of less than 2.5%.
Run an isocratic run of 5.5 min under the mobile phase conditions specified above. Inject twice the sample prepared as specified above.
Run a standard every 20 injections and verify that the area of the standard is within the average for each standard plus or minus 2.5%.
The HPLC method gives the result in mg/g. Calculate:
mg/strip=mg/g×weight of the strip
Write the answer in the HPLC notebook and attach a copy of the chromatogram to the batch record.

### Monographs

Melatonin

Description

Melatonin is the principal hormone of the vertebrate pineal gland, and it is also produced by extra-pineal tissues in amphibians. It is found in plants as well, but at much lower concentrations than in animals. This hormone is involved in setting the timing (entrainment) of mammalian circadian rhythms, as well as regulating seasonal responses to changes in day length in seasonally breeding mammals so called photoperiodic responses. Photoperiodic responses include changes in reproductive status, behavior and body weight. Seasonal effects on reproduction in humans are subtle, and the role of melatonin here, if any, is unclear. Recently, melatonin supplementation has become popular as a possible aid for sleep disorders among other things.

Melatonin is synthesized endogenously by the pinealocytes of the pineal gland. The essential amino acid L-tryptophan is a precursor in the synthesis of melatonin. In this synthesis, L-tryptophan first gets metabolized to 5-hydroxytryptophan from which 5-hydroxytryptamine, also known as serotonin, is made. 5-hydroxytryptamine is converted to melatonin in a two-step process, occurring mainly in the pineal gland.

Melatonin is also known as N-acetyl-5-methoxytryptamine and N-[2-(5-Methoxy-1H-indol-3-yl)ethyl]acetamide. The structural formula is:

Melatonin is a solid, lipophilic, hydrophobic substance, which is available as a supplement in synthetic form. Melatonin derived from the pineal glands of beef cattle is also marketed.

Actions and Pharmacology

Actions

Supplemental melatonin may have a hypnotic action. It may also have antioxidant and anti-apoptotic activity.

Mechanism of Action

Melatonin is derived in pinealocytes from L-tryptophan. 5-hydroxytryptamine or serotonin is an intermediate in the biosynthetic process. The rate limiting step in the synthesis of melatonin is the n-acetylation of the 5-hydroxytryptamine by the enzyme arylalkylamine N-acetyltransferase (AA-NAT). Melatonin synthesis displays a circadian rhythm that is reflected in serum melatonin levels. The rhythm is generated by a circadian clock located in the suprachiasmatic nucleus (SCN) of the hypothalamus. The SCN clock is set to the 24 hour day by the natural light-dark cycle. Light signals through a direct retinal pathway to the SCN. The SCN clock sends circadian signals over a neural pathway to the pineal gland. This drives rhythmic melatonin synthesis. The neural input to the gland is norepinephrine, and the output is melatonin. Specifically, the rhythm of the enzyme AA-NAT is under SCN control, with the resulting melatonin rhythm characterized by high levels at night. Thus, the synthesis and release of melatonin are stimulated by darkness and inhibited by light.

The effects of hormones are typically mediated through receptors. Two forms of high-affinity melatonin receptors and one form of a low-affinity receptor have been identified.

The high-affinity ML1 receptors are designated Mel1a and Mel1b. The low-affinity receptor is designated ML2.

The Mel1a receptor is expressed in the SCN and in the hypophyseal pars tuberalis. The SCN is the putative site of circadian action of melatonin, and the hypophyseal pars tuberalis is the putative site of its reproductive effects. The Mel1b receptor is expressed mainly in the retina. The ML1 melatonin receptors belong to the family of guanadine triphosphate-binding proteins or G protein-coupled receptors. Activation of the ML1 receptors results in inhibition of adenylate cyclase activity in target cells.

The distribution of the ML2 receptors has not yet been determined. These receptors are coupled to the stimulation of phosphoinositide hydrolysis.

In summary, melatonin is a hormone that has biological effects and that signals through a family of G protein-coupled receptors.

Melatonin has antioxidant activity. However, this activity is found only with very high pharmaceutical doses of this substance. The most significant antioxidant activity of melatonin appears to be its ability to inhibit metal ion-catalyzed oxidation processes, specifically the Fenton reaction.

Melatonin has been found to have anti-apoptotic activity in the thymus. Melatonin inhibits apoptosis in the thymus as well as in cultured dexamethasone-treated thymocytes (a standard model for the study of apoptosis). It is thought to do so by down-regulating the glucocorticoid receptor.

The mechanism of action of supplemental melatonin is speculative. The putative effect of melatonin as a hypnotic may be accounted for by receptor-mediated action on the limbic system. Pharmacologic doses of melatonin may produce a hypothermic effect, which may also play a role in its hypnotic effect.

Pharmacokinetics

The absorption and bioavailability of melatonin varies widely. Melatonin is absorbed from the small intestine and is transported by the portal circulation to the liver. Variable amounts of ingested melatonin are metabolized in the liver to 6-hydroxymelatonin. After conjugation with sulfuric or glucuronic acid, it is excreted by the kidneys.

Nonmetabolized melatonin is transported via the systemic circulation to various tissues in the body. Serum half-life of ingested melatonin is approximately 35 to 50 minutes. If melatonin causes drowsiness, this effect occurs about 30 minutes after ingestion and lasts for at least an hour. Melatonin given in the early evening appears to advance the nighttime peak of melatonin secretion by about three hours. Ingested melatonin that did not undergo first-pass metabolism in the liver is eventually metabolized, mainly in the liver, by hydroxylation to 6-hydroxymelatonin. After conjugation with sulfuric or glucuronic acid, it is excreted by the kidneys. A single nighttime dose is cleared by the following morning. With chronic dosing, however, some lipid storage occurs.

Indications and Usage

Melatonin may be indicated for some forms of insomnia and other sleep disturbances. Research results are mixed with respect to claims that melatonin can abolish some of the symptoms of jet lag. Use of the supplement in cancer and immune disorders is unsupported by current research; there are some promising findings, but they are very preliminary. There is no evidence to substantiate claims that melatonin can delay aging, be useful in cardiovascular disease, depression, seasonal affective disorder or sexual dysfunction.

Research Summary

Numerous studies, many of them well-designed, suggest that supplemental melatonin can be effective in some sleep disorders, principally insomnia. These studies show that, in doses that raise serum melatonin levels to those that approximate normal nocturnal levels, sleep can be induced and sustained in some. Through its effects on circadian rhythms and possibly through an induced hypothermic effect, melatonin, in doses administered at carefully timed intervals, may be able to normalize various sleep disorders, such as those sometimes experienced by shift workers, and thus diminish fatigue.

The complexity of appropriate timing and dosage, however, has prompted some researchers to caution against melatonin use for sleep disturbance outside of laboratory settings or without medical supervision at least until more research sheds further light on these issues. Even marginal drowsiness or lack of mental alertness could prove hazardous for some shift workers, for example.

In addition, a cautionary note has recently been issued with respect to the use of melatonin to treat sleep disturbances in children with neurologic disorders. Six such children, aged nine months to 18 years, were given 5 milligrams of melatonin at bedtime in as effort to treat their sleep disorder. Quality and quantity of sleep quickly increased in five of the six children. But in four of the subjects, all of whom had a prior history of seizures, incidence of seizures increased while taking melatonin. Discontinuance of the supplements led to seizure-incidence returning to pre-supplementation levels. But resumption of melatonin supplementation, this time at a reduced level of 1 milligram doses, again caused an increase in seizures, and the study was halted. Some criticized these researchers for using inappropriately high doses, but the typical dose range in studies of melatonin's effects on sleep disturbance has been 0.3 milligrams to 5 milligrams, with 2 to 3 milligrams commonly being used. Clearly, more research is needed before melatonin can safely be recommended for use in individuals, whether children or adults, with seizure history. In addition, safety data, in general, is lacking for use of this supplement, particularly for long-term use. Certainly, if more research better defines the proper use of melatonin in sleep disturbances, the supplement might make a significant contribution considering that many sleep-deprived individuals become dependent upon benzodiazepine and other sedating drugs with potentially serious adverse effects in search of insomnia relief.

This point was made in a recent well-designed study that tested the effects of melatonin (2 milligrams daily) in a controlled release formula against placebo. During the course of the study, 34 long-term users of benzodiazepine were encouraged to reduce their benzodiazepine dosage incrementally. The goal was complete discontinuance during weeks five and six. The study proceeded double-blind through the six weeks of period one and then single-blind through the six weeks of period two, during which all subjects received melatonin and efforts to discontinue benzodiazepine resumed.

At the end of the study, 14 of 18 subjects who received melatonin in period one had completely discontinued benzodiazepine use; only four of 16 in the placebo group achieved this goal. An additional six subjects in the placebo group achieved complete discontinuance of benzodiazepine in period two. Sleep quality scores were significantly higher for the melatonin group than for the placebo group. A six-month post-study followup showed that 19 of the 24 subjects who discontinued benzodiazepine therapy continued to maintain good sleep quality. These subjects continued to use melatonin after the study ended and they did not resume use of benzodiazepine.

The use of melatonin to help alleviate some of the symptoms of jet lag has produced mixed results in trials to date. Often some benefit has been noted, but many studies have been criticized for being small and poorly designed. In the largest controlled trial to date, researchers recently reported that melatonin exerts no beneficial physiological effect on jet lag. Melatonin was tested against placebo in two doses and with different administration times. No melatonin regimen was superior to placebo.

Claims that melatonin can be used to prevent or treat cancer or immune dysfunction are unsupported by current research. There is some very preliminary data suggesting some beneficial effects in animal models and in in vitro studies. A small amount of clinical work has been done, and more seems warranted.

Claims that melatonin can favorably influence lipids, lower blood pressure and help prevent heart attacks are entirely baseless, as are claims that it can correct sexual dysfunction or otherwise enhance sexual performance. It has demonstrated no effect in seasonal affective disorder and, rather than help dispel depression it has been reported to cause or worsen it in some cases.

The sensational claim that melatonin dramatically delays aging is similarly without foundation. The claim was based, generally, on the long-held belief that endogenous melatonin secretion diminishes with age and, specifically, upon a single mouse study that has been criticized as seriously flawed by several researchers.

The idea that levels of serotonin fall with age was refuted in a recent study of 34 healthy subjects aged 65 to 81 in whom plasma melatonin concentrations were compared with those of a younger subject group (98 healthy individuals aged 18 to 30). No significant difference was noted between the two groups. The researchers have cautioned against the use of melatonin by the elderly, particularly since many of them may be using a variety of prescription drugs for which interactions with melatonin are unknown and could be potentially hazardous.

Contraindications, Precautions and Adverse Reactions

Contraindications

None known.

Precautions

Use of melatonin in children, pregnant women and nursing mothers is not advised. Adverse reactions of supplemental melatonin include depression. Those who suffer from depression are advised against taking melatonin.

Because melatonin may cause both nighttime and daytime drowsiness, those who operate hazardous machinery are advised against taking melatonin.

Large doses of melatonin (not recommended) have been shown to inhibit ovulation. Women who are trying to conceive should avoid melatonin.

Melatonin use in some children with seizure disorders leads to increased seizure activity. Those with seizure disorders, both children and adults, should avoid melatonin supplements.

Those over 65 years old who take any sedating medications or herbs, or who use alcohol, should exercise caution in the use of melatonin.

Adverse Reactions

Adverse reactions associated with melatonin include stomach discomfort, morning grogginess, daytime “hangover,” feeling of a “heavy head,” depression, psychotic episodes (in combination with fluoxetine), headache, lethargy, fragmented disorientation, amnesia, inhibition of fertility, increased seizure activity, suppression of male sexual drive, hypothermia, retinal damage, gynecomastia and low sperm count. Typically, these reports are related to high doses. However, adverse effects have been reported and can occur with low doses as well.

Interactions

Drugs

Aspirin and other NSAIDs may lead to decreased melatonin levels.

The bioavailability of oral melatonin is increased by coadministration of fluvoxamine.

This is believed due to inhibition of the elimination of melatonin.

Beta blockers may lead to decreased melatonin levels.

A psychotic episode has been reported associated with the use of melatonin in a subject taking the antidepressant fluoxetine.

There is a report of melatonin augmenting the antitumor effect of interleukin-2.

There is a report of melatonin enhancing the activity of the anti-Mycobacterium tuberculosis drug, isoniazid.

Melatonin and progestin combinations can be additive in inhibiting ovarian function in women.

Use of melatonin with benzodiazepenes, sedating antihistamines, sedating antidepressants and other sedating drugs may cause additive sedation and increase incidence of adverse effects.

Use of melatonin with corticosteroids may interfere with the efficacy of the corticosteroids.

Herbs

Use of melatonin with valerian or kava kava may lead to additive sedation.

Nutritional Supplements

Use of melatonin with 5-hydroxytryptophan may lead to additive sedation.

Alcohol

Use of melatonin with alcohol may lead to additive sedation.

Food

No interactions are known.

Overdosage

None known. No apparent serious consequences have been reported in those taking up to 24 grams daily of melatonin for one month, though such doses are not recommended.

Dosage and Administration

Those who use melatonin supplements for sleep disturbance or jet lag usually take no more than 0.3 milligrams to 3 milligrams at bedtime for short periods of time (no longer than two weeks). Higher doses and dosing for longer periods of time requires medical supervision. As with all nutritional supplements, the physician must know if his or her patient is taking melatonin. Melatonin supplements derived from animals should be avoided.

Kava-Kava

Latin name: Piper methysticum

A Remedy For
Anxiety
Insomnia
Nervousness

In the past, Kava Kava has been taken for a host of ailments on which it has no appreciable effect, including asthma, arthritis, indigestion, cystitis, syphilis, and gonorrhea. For tension and sleeplessness, however, it is now considered a proven remedy.

What It Is; Why It Works

One of the “new” herbs that have recently gained considerable media attention, Kava Kava has actually been around for centuries in the South Seas, where it's used as a ceremonial beverage. The plant's fleshy underground stem is mildly intoxicating when chewed. Prepared as a nonalcoholic drink, it is said to foster a sense of contentment and well-being, while sharpening the mind, memory, and senses.

Research shows that the active ingredients in Kava Kava (kava pyrones) do in fact have a calming, sedative effect. They also appear to relax the muscles, relieve spasms, and prevent convulsions. At least two scientific studies have confirmed the herb's ability to significantly reduce symptoms of anxiety. In a third study, researchers rated it as effective as prescription tranquilizers.

Avoid If . . .

Do not use Kava Kava if you are pregnant or nursing. Also avoid it if you have a depressive disorder; it can deepen a depressed mood.

Special Cautions

When first taking Kava Kava, you may notice a slightly tired feeling in the mornings.

In rare cases, Kava Kava can cause an allergic reaction, a slight yellowing of the skin, gastrointestinal complaints, impaired or abnormal movement, loss of balance, pupil dilation, and difficulty focusing. Because of the possibility of visual disturbances, drive with caution while using this herb.

High doses of the herb have been known to trigger hepatitis. Heavy long-term use can also cause an unusual scaly rash, and may lead to unwanted weight loss. Do not take this herb for more than 3 months without consulting a physician.

Possible Drug Interactions

Do not take Kava Kava when using other substances that act on the brain, such as alcohol, barbiturates, or other mood-altering drugs. It may increase their effect. Be especially wary of taking it with the tranquilizer Xanax; the combination has caused coma. Kava Kava also has an antagonistic effect on dopamine. If you are taking a levodopa-based medication for Parkinson's disease, avoid this herb.

Special Information If You are Pregnant or Breastfeeding

Remember, Kava Kava should be avoided during pregnancy and nursing.

How to Prepare

Commercial extracts are the predominant form of Kava Kava. The crushed root can also be used.

Typical Dosage

Daily doses delivering between 50 and 240 milligrams of the active ingredients are the customary recommendation. Commercial capsules containing between 150 and 300 milligrams of root extract may be taken twice a day. Because the potency of commercial preparations may vary, follow the manufacturer's directions whenever available.

Overdosage

An overdose is usually signaled by a lack of coordination, followed by tiredness and a tendency to sleep. If you suspect an overdose, seek medical attention immediately.

Huperzine-A

Description

Huperzine A is a plant alkaloid derived from the Chinese club moss plant, Huperzia serrata, which is a member of the Lycopodium species. Huperzia serrata has been used in Chinese folk medicine for the treatment of fevers and inflammation.

Huperzine A has been found to have acetylcholinesterase activity. Huperzine B, also derived from Huperzia serrata, is a much less potent acetylcholinesterase inhibitor. Natural huperzine A is a chiral molecule also called L-huperzine A or (−)-huperzine A. Synthetic huperzine A is a racemic mixture called (±)-huperzine A. Huperzine A is also known as HUP, hup A and selagine. In Chinese medicine, the extract of Huperzia serrata is known as Chien Tseng Ta and shuangyiping. Huperzine A derivatives are being developed for pharmaceutical application.

Actions and Pharmacology

Actions

Huperzine A may have cognition-enhancing activity in some.

Mechanism of Action

Alzheimer's disease is a neurodegenerative disorder associated with neuritic plaques that affect the cerebral cortex, amygdala and hippocampus. There is also neurotransmission damage in the brain. One of the major functional deficits in Alzheimer's disease is a hypofunction of cholinergic neurons. This leads to the cholinergic hypothesis of Alzheimer's disease and the rationale for strategies to increase acetylcholine in the brains of Alzheimer's disease patients. Two FDA-approved drugs for the treatment of Alzheimer's disease, tacrine and donepezil, are acetylcholinesterase inhibitors.

Huperzine A is also an acetylcholinesterase inhibitor and has been found to increase acetylcholine levels in the rat brain following its administration. It also increases norepinephrine and dopamine, but not serotonin levels. The natural L or (−)-huperzine A is approximately three times more potent than the racemic or (±)-huperzine A in vitro.

Pharmacokinetics

There are limited pharmacokinetic studies with huperzine A. It appears that huperzine A is rapidly absorbed from the gastrointestinal tract and transported to the liver via the portal circulation. Some first-pass metabolism takes place in the liver, and huperzine A and its metabolites are distributed widely in the body, including to the brain. Following ingestion, the time to reach peak blood level is approximately 80 minutes.

Indications and Usage

Huperzine A has potent pharmacological effects and, particularly since long-term safety has not been determined, it should only be used with medical supervision. It may have some effectiveness in Alzheimer's disease and age-related memory impairment. It has been used to treat fever and some inflammatory disorders, but there is no credible scientific evidence to support these uses.

Research Summary

Numerous studies, most of them from China, suggest that huperzine A may be as effective as the drugs tacrine and donepezil in Alzheimer's disease. This is not so surprising since in vitro and animal model tests have demonstrated that huperzine A effectively inhibits acetylcholinesterase, an enzyme that catalyzes acetylcholine breakdown. Tacrine and donepezil work in the same way to conserve acetylcholine in the brain—the mode by which they presumptively improve memory and cognition in those with Alzheimer's and age-related cognitive impairment. Huperzine A may prove superior to tacrine (dose-limited due to its hepatotoxicity) if long-range studies, yet to be conducted, demonstrate its safety.

In one double-blind, randomized study, huperzine A, in injectable form, was tested against a saline control in 56 patients with multi-infarct dementia or senile dementia and in 104 patients with senile and pre-senile simple memory disorders. Huperzine A produced significant positive effects as measured by the Wechsler Memory Scale. Dizziness was experienced by a few of the huperzine A-treated patients.

In another study, this one multicenter, double-blind, placebo-controlled and randomized, 50 subjects with Alzheimer's disease were given huperzine A or placebo for eight weeks. Significant improvement was noted in 58 percent of the patients in terms of memory, cognitive and behavioral functions. Research is ongoing.

Contraindications, Precautions and Adverse Reactions

Contraindications

None known.

Precautions

Huperzine A should be avoided by children, pregnant women and nursing mothers.

Because of possible adverse effects in those with seizure disorders, cardiac arrhythmias and asthma, those with these disorders should avoid huperzine A. Those with irritable bowel disease, inflammatory bowel disease and malabsorption syndromes should avoid huperzine A.

Adverse Reactions

Adverse effects reported with huperzine A include gastrointestinal effects, such as nausea and diarrhea, sweating, blurred vision, fasciculations and dizziness. Possible adverse effects include vomiting, cramping, bronchospasm, bradycardia, arrhythmias, seizures, urinary incontinence, increased urination and hypersalivation.

Interactions

Drugs

Acetylcholinesterase Inhibitors: Use of huperzine A along with the acetylcholinesterase inhibitors donepezil or tacrine may produce additive effects, including additive adverse effects. Other acetylcholinesterase inhibitors include neostigmine, physostigmine and pyridostigmine, and use of these agents along with huperzine A may produce additive effects, including additive adverse effects.

Cholinergic Drugs Use of huperzine A along with cholinergic drugs, such as bethanechol, may produce additive effects, including additive adverse effects.

Nutritional Supplements

Use of huperzine A with choline, phosphatidylcholine, CDP-choline and L-alpha-glycerylphosphorylcholine hypothetically might produce additive effects, including additive adverse effects.

Overdosage

There are no reports of overdosage with huperzine A.

Dosage and Administration

There are various forms of huperzine A available, including extracts of Huperzia serrata, natural (−)-huperzine A and synthetic racemic (±)-huperzine A. Natural (−)-huperzine A is approximately three times more potent than the synthetic racemic mixture. The doses of natural (−)-huperzine A used in clinical studies ranged from 60 micrograms to 200 micrograms daily. Huperzine A should only be used with a physician's recommendation and monitoring.

### Clinical Studies of Active Ingredients

Melatonin

Different criteria of sleep latency and the effect of melatonin on sleep consolidation. Pinto L R Jr, Seabra Mde L, Tufik S.

Department of Psychobiology, Universidade Federal de São Paulo, São Paulo, Brazil. luciano@psicobio.epm.br

Objectives: Since there is no consensus definition of sleep onset, we studied different aspects of initial sleep periods in healthy volunteers taking melatonin. Two criteria for sleep latency were used: 10 minutes of uninterrupted sleep and 1.5 minutes of stage 1 sleep.

Participants: Forty healthy male volunteers (mean age 28+−5 years) were assigned to 2 groups: 30 ingested melatonin and 10 placebos.

Design: All volunteers underwent an initial polysomnogram (baseline) after a 1-night adaptation period. The next day, the placebo or a 10-mg dose of melatonin was administered for 28 days, 1 hour before sleep time, in double-blind fashion. The second polysomnogram was recorded on day

Setting: Sleep laboratory

Results: Chronic melatonin administration led to a significant reduction in sleep latency, using only the criterion 10 minutes of uninterrupted sleep. This effect suggests that melatonin may have a hypnotic effect, and the use of melatonin may lead to better sleep consolidation.

Conclusions: These results show differences that have clinical implications, since the criteria used to diagnose initial insomnia were based on sleep onset.

Cognitive effects of exogenous melatonin administration in elderly persons: a pilot study.

Peck J S, LeGoff D B, Ahmed I, Goebert D.

University of Hawaii, John A. Burns School of Medicine, Department of Psychiatry, 1356 Lusitania Street, 4th Floor, Honolulu, Hi. 96813, USA.

Objective: Given that circadian rhythm disruption is associated with impairments in cognitive performance similar to those found in age-related cognitive decline, the authors investigated whether exogenous melatonin administration would improve cognitive functioning in healthy elderly subjects.

Methods: This double-blind, placebo-controlled pilot study assigned 26 healthy elderly subjects to receive either melatonin 1 mg or placebo nightly for 4 weeks. Participants completed a sleep questionnaire and a battery of cognitive tests at baseline and at 4 weeks. Results: Melatonin administration improved reported morning “restedness” and sleep latency after nocturnal awakening, and also improved scores on the California Verbal Learning Test-interference subtest.

Conclusions: Melatonin administration at a dose of 1 mg nightly may be effective in improving certain aspects of cognitive functioning and subjective reports of sleep quality in elderly subjects. It may prove to be a useful therapeutic agent in the treatment of age-related cognitive decline.

Melatonin improves health status and sleep in children with idiopathic chronic sleep-onset insomnia: a randomized placebo-controlled trial.

Smits M G, van Stel H F, van der Heijden K, Meijer A M, Coenen A M, Kerkhof G A. Sleep Centre, Hospital Gelderse Vallei, Willy Brandtlaan 10, Box 9025, 6710 HN Ede, the Netherlands. smitsm@zgv.nl

Objective: To investigate the effect of melatonin treatment on health status and sleep in children with idiopathic sleep-onset insomnia.

Method: A randomized, double-blind, placebo-controlled trial was conducted in a Dutch sleep center, involving 62 children, 6 to 12 years of age, who suffered more than 1 year from idiopathic chronic sleep-onset insomnia. Patients received either 5 mg melatonin or placebo at 7 pm. The study consisted of a 1-week baseline period, followed by a 4-week treatment. Health status was measured with the RAND General Health Rating Index (RAND-GHRI) and Functional Status II (FS-II) questionnaires. Lights-off time, sleep onset, and wake-up time were recorded in a diary, and endogenous dim light melatonin onset was measured in saliva.

Results: The total scores of the RAND-GHRI and FS-II improved significantly more during melatonin treatment compared to placebo. The magnitude of change was much higher in the melatonin group than in the placebo group, with standardized response means for the RAND-GHRI of 0.69 versus 0.07 and for the FS-II of 1.61 versus 0.64. Melatonin treatment also significantly advanced sleep onset by 57 minutes, sleep offset by 9 minutes, and melatonin onset by 82 minutes, and decreased sleep latency by 17 minutes. Lights-off time and total sleep time did not change.

Conclusions: Melatonin improves health status and advances the sleep-wake rhythm in children with idiopathic chronic sleep-onset insomnia.

The effects of melatonin on tinnitus and sleep.

Megwalu U C, Finnell J E, Piccirillo J F.

Clinical Outcomes Research Office, Department of Otolaryngology-Head and Neck Surgery, Washington University School of Medicine, St. Louis, Mo., USA.

Goal: To determine if melatonin improves tinnitus and if this improvement is related to improvement in sleep.

Study Design and Setting: Prospective open-label study of 24 patients with tinnitus. The patients took 3 mg of melatonin per day for 4 weeks, followed by 4 weeks of observation. The Tinnitus Handicap Inventory (THI) and the Pittsburgh Sleep Quality Index (PSQI) were administered.

Results: The mean THI score decreased significantly between weeks 0 and 4, and between weeks 0 and 8. The mean PSQI significantly decreased between weeks 0 and 4 (P<0.0001), and between weeks 0 and 8 (P=0.0003). The change in PSQI was significantly associated with the change in THI between weeks 0 and 4. The change in PSQI was not significantly associated with the change in THI between weeks 0 and 8. The change in the PSQI in the first 4 weeks was associated with the initial PSQI. There was no association between the initial THI and the change in the THI in the first 4 weeks.

Conclusion: Melatonin use is associated with improvement of tinnitus and sleep. There was an association between the amount of improvement in sleep and tinnitus. The impact of melatonin on sleep was greatest among patients with the worst sleep quality, but its impact on tinnitus was not associated with the severity of the tinnitus.

Significance: Melatonin may be a safe treatment for patients with idiopathic tinnitus, especially those with sleep disturbance due to tinnitus.

Melatonin in schizophrenic outpatients with insomnia: a double-blind, placebo-controlled study.

Suresh Kumar P N, Andrade C, Bhakta S G, Singh N M.

Institute of Mental Health and Neurosciences, Kozhikode.

Background: Low nighttime levels of melatonin have been demonstrated in patients with insomnia, and melatonin has been shown to have hypnotic properties in some groups of such subjects. Low melatonin levels have also been observed in patients with schizophrenia; however, there is little literature on the efficacy of exogenous melatonin in treating insomnia associated with schizophrenia.

Method: Stable DSM-IV schizophrenic outpatients (N=40) with initial insomnia of at least 2 weeks' duration were randomly assigned to augment their current medications with either flexibly dosed melatonin (3-12 mg/night; N=20) or placebo (N=20). By use of a questionnaire, double-blind assessments of aspects of sleep functioning were obtained daily across the next 15 days. The study was conducted between March and December 2002.

Results: The modal stable dose of melatonin was 3 mg. Relative to placebo, melatonin significantly improved the quality and depth of nighttime sleep, reduced the number of nighttime awakenings, and increased the duration of sleep without producing a morning hangover (p<0.05). Subjectively, melatonin also significantly reduced sleep-onset latency, heightened freshness on awakening, improved mood, and improved daytime functioning (p<0.05).

Conclusion: Melatonin may be a useful short-term hypnotic for schizophrenic patients with insomnia. Melatonin could be considered for patients in whom conventional hypnotic drug therapy or higher sedative antipsychotic drug doses may be problematic.

Melatonin Improves Sleep in Asthma

A Randomized, Double-blind, Placebo-controlled Study

Francineide L. Campos, Francisco P. da Silva-Júnior, Veralice M. S. de Bruin and Pedro F. C. de Bruin

Department of Pharmacy and Department of Medicine, Universidade Federal do Ceará, Fortaleza, Brazil

Correspondence and requests for reprints should be addressed to Pedro F. C. de Bruin, M. D., Ph.D., Department of Medicine, Universidade Federal do Ceara, Rua Prof. Costa Mendes, 1608-4° andar, Fortaleza, C E, Brazil. E-mail: pfelipe@superig.com.br

Disturbed sleep is common in asthma. Melatonin has sleep-inducing activity and reportedly affects smooth muscle tone and inflammation. The aim of this study was to evaluate the effect of melatonin on sleep in patients with mild and moderate asthma. This was a randomized, double-blind, placebo-controlled study. Twenty-two consecutive women with asthma were randomized to receive melatonin 3 mg (n=12) or placebo (n=10) for 4 weeks. Sleep quality and daytime somnolence were assessed by the Pittsburgh Sleep Quality Index and the Epworth Sleepiness Scale, respectively. Pulmonary function was assessed by spirometry. Use of relief medication, asthma symptoms, and morning and evening peak expiratory flow rate were recorded daily. Melatonin treatment significantly improved subjective sleep quality, as compared with placebo (p=0.04). No significant difference in asthma symptoms, use of relief medication and daily peak expiratory flow rate was found between groups. We conclude that melatonin can improve sleep in patients with asthma.

Further studies looking into long-term effects of melatonin on airway inflammation and bronchial hyperresponsiveness are needed before melatonin can be recommended in patients with asthma.

Melatonin enhances cortisol levels in aged but not young women

A Cagnacci, R Soldani, and SS Yen

Department of Reproductive Medicine, School of Medicine, University of California, San Diego, La Jolla, USA.

In spite of animal data showing an effect of melatonin in the regulation of the hypothalamus-pituitary-adrenal (HPA) axis, no effect of melatonin on cortisol has been evidenced in young men. Gender and aging are believed to influence the regulation of the HPA axis, and may thus modulate the melatonin effect on cortisol. In this study we investigated whether an effect of melatonin on cortisol can be observed in women of different age. Six young women in early follicular phase (22-32 years; EFW) and eight aged women in postmenopause (54-62 years; PMW) were studied. At 08.00 h on two consecutive days each woman received, randomly and in double-blind fashion, a pill of placebo or melatonin (100 mg). Serum levels of melatonin and cortisol were evaluated at 20-min intervals for 48 h. In comparison to EFW, PMW showed an earlier onset of nocturnal melatonin (p<0.05) and cortisol rise (p<0.01) and higher cortisol levels at lunch (p<0.05) and early evening (p<0.01). Melatonin administration did not modify serum cortisol levels in EFW but elicited a marked increase of daytime cortisol levels in PMW (p<0.02). The present data reveal that in aged PMW the cortisol levels are enhanced at selected circadian times and are stimulated by melatonin.

Several studies have shown that ingestion of melatonin can decrease sleep latency (make you fall asleep faster), induce sleep, increase sleepiness, increase sleep efficiency, and increase sleep duration.

Reference: Brzezinski, A. Melatonin in Humans. The New England Journal of Medicine. 1997. 336(3), 186-195.

Melatonin has also been shown to reduce the symptoms of jet lag. One meta-analysis demonstrated that travelers crossing five or more time zones—especially in an eastward direction—can effectively use melatonin to reduce or prevent jet lag. This is particularly true for those individuals who have experienced jet lag on previous journeys, and using melatonin may prove beneficial for such individuals even when experiencing a less drastic time change.

Studies have also found that melatonin may be beneficial in helping blind people to establish a normal-sleeping pattern.

The use melatonin in the treatment of sleep disorders appears to be less effective. A recent meta-analysis found that the use of melatonin is not effective for treating either primary or secondary sleep disorders. The study did find a moderate amount of evidence for the efficacy of melatonin in treating delayed sleep phase syndrome. However, this study contradicted previous studies in its finding of no evidence to support the use of melatonin in alleviating jet lag and shift-work sleep disturbances. (Reference Buscemi, N et al. BMJ 2006:332:385-393.)

Kava-Kava Extract

Effects of kava-kava extract on the sleep-wake cycle in sleep-disturbed rats. Shinomiya K, Inoue T, Utsu Y, Tokunaga S, Masuoka T, Ohmori A, Kamei C. Department of Pharmacology, Faculty of Pharmaceutical Sciences, Okayama University, Tsushima-naka 1-1-1, Okayama, 700-8530, Japan.

Rationale: Kava-kava extract may be useful as an herbal medicine for treatment of insomnia and anxiety.

Objectives: The present study was undertaken to investigate the effects of kava-kava extract on the sleep-wake cycle in comparison with that of flunitrazepam using sleep-disturbed rats.

Methods: Electrodes for measurement of electroencephalogram (EEG) and electromyogram (EMG) were implanted into the frontal cortex and the dorsal neck muscle of rats. EEG and EMG were recorded with an electroencephalogram. SleepSign ver. 2.0 was used for EEG and EMG analysis. Total times of wakefulness, non-rapid eye movement (non-REM) and REM sleep were measured from 09:00 to 15:00.

Results: A significant shortening of the sleep latency in sleep-disturbed rats was observed following the administration of kava-kava extract at a dose of 300 mg/kg, while no effects were observed on the total waking and non-REM sleep time. On the other hand, flunitrazepam showed a significant shortening in sleep latency, decrease in total waking time and increase in total non-REM sleep time. Although the effects of flunitrazepam were antagonized by the benzodiazepine receptor antagonist flumazenil, the effect of kava-kava extract was not antagonized by flumazenil. Kava-kava extract showed a significant increase in delta activity during non-REM sleep in sleep-disturbed rats, whereas a significant decrease in delta power during non-REM sleep was observed with flunitrazepam. Flumazenil caused no significant effect on the changes in delta activity induced by both kava-kava extract and flunitrazepam.

Conclusions: Kava-kava extract is an herbal medicine having not only hypnotic effects, but also sleep quality-enhancement effects.

Clinical efficacy of kava extract WS 1490 in sleep disturbances associated with anxiety disorders. Results of a multicenter, randomized, placebo-controlled, double-blind clinical trial.

Lehrl S.

Clinic and Polyclinic for Psychiatry and Psychotherapy of the University of Erlangen-Nuremberg,

Department of Medical Psychology and Medical Sociology, Schwabachanlage 6, D-91054, Erlangen, Germany.

Background: The aim of the present trial was to investigate the efficacy and safety of kava special extract WS 1490 in patients with sleep disturbances associated with anxiety, tension and restlessness states of non-psychotic origin.

Methods: In a multicenter, randomized, double-blind clinical study, 61 patients received daily doses of 200 mg WS 1490 or placebo over a period of 4 weeks. Efficacy was measured by the sleep questionnaire SF-B, the Hamilton Anxiety Scale (HAMA), the Bf-S self-rating scale of well-being and the Clinical Global Impressions (CGI) scale.

Results: The confirmatory analysis of the two primary efficacy variables, the differences of sleep questionnaire SF-B sub-scores ‘Quality of sleep’ and ‘Recuperative effect after sleep’ after 4 weeks of double-blind treatment compared to baseline, demonstrated statistically significant group differences in favor of kava extract WS 1490 (P=0.007 and P=0.018, respectively). Superior effects of kava extract were also present in the HAMA psychic anxiety sub-score (P=0.002). More pronounced effects with respect to the self-rating of well-being and the global clinical evaluation also indicated superior therapeutic efficacy of kava extract. Safety and tolerability were good, with no drug-related adverse events or changes in clinical or laboratory parameters.

Conclusions: We conclude that sleep disturbances associated with non-psychotic anxiety disorders can be effectively and safely treated with kava extract WS 1490.

Kava extract for treating anxiety.

Pittler M H, Ernst E.

Department of Complementary Medicine, University of Exeter, 25 Victoria Park Road, Exeter, Devon, UK, EX2 4NT. M. H. Pittler@ex.ac.uk

Background: Constraints on resources and time often render treatments for anxiety such as psychological interventions impracticable, while synthetic anxiolytic drugs are effective, but are often burdened with adverse events. Options which are effective and safe would be of considerable interest and a welcome addition to the therapeutic repertoire.

Objectives: To assess the effectiveness and safety as reported in rigorous clinical trials of kava extract compared with placebo for treating anxiety.

Search Strategy: All publications describing (or which might describe) randomised, double-blind, placebo-controlled trials of kava extract for anxiety were sought through electronic searches on EMBASE, MEDLINE, AMED (British Library), CISCOM (Research Council for Complementary Medicine, London), Central/CCTR and CCDANCTR. The search terms that were used were kava, kawa, kavain, Piper methysticum and Rauschpfeffer (German common name for Piper methysticum). Additionally, manufacturers of kava preparations and experts on the subject were contacted and asked to contribute published and unpublished material. Hand-searches of relevant medical journals (Erfahrungsheilkunde 1996-2002, Forsch Komplementärmed Klass Naturheilkd 1994-2002, Phytomed 1994-2002, Alt Comp Ther 1995-2002), conference proceedings (e.g. FACT—Focus on Alternative and Complementary Therapies 1996-2002) and our own files were conducted. The searches were updated to August 2002. No restrictions regarding the language of publication were imposed.

Selection Criteria To be included studies were required to be randomised, controlled trials (RCTs), i.e. trials with a randomised generation of allocation sequences, and conducted placebo-controlled and double-blind, i.e. trials with blinding of patients and care providers. Trials using oral preparations containing kava extract as the only component (mono-preparation) were considered. Trials using single constituents of kava extract alone, assessing kava extract as one of several active components in a combination preparation or as a part of a combination therapy were excluded.

Data Collection and Analysis: Data were extracted systematically according to patient characteristics, interventions and results. Methodological quality of all trials was evaluated using the standard scoring system developed by Jadad and colleagues. The screening of studies, selection, data extraction, validation and the assessment of methodological quality were performed independently by the two reviewers. Disagreements in the evaluation of individual trials were resolved through discussion.

Main Results Eleven trials with a total of 645 participants met the inclusion criteria. The meta-analysis of six studies using the total score on the Hamilton Anxiety scale as a common outcome measure suggests a significant reduction in patients receiving kava extract compared with patients receiving placebo (weighted mean difference: 5.0, 95% confidence interval: 1.1 to 8.8; p=0.01; n=345). Adverse events as reported in the reviewed trials were mild, transient and infrequent.

Reviewer's Conclusions: Compared with placebo, kava extract appears to be an effective symptomatic treatment option for anxiety. The data available from the reviewed studies suggest that kava is relatively safe for short-term treatment (1 to 24 weeks), although more information is required. Further rigorous investigations, particularly into the long-term safety profile of kava are warranted.

Kava-Kava administration reduces anxiety in perimenopausal women.

Cagnacci A, Arangino S, Renzi A, Zanni A L, Malmusi S, Volpe A. Institute of Obstetrics and Gynecology, University of Modena, Via del Pozzo 71, 41100, Modena, Italy. cagnacci@unime.it

Objective: Disturbances of mood, such as anxiety and depression, increase in the perimenopausal period. Hormone replacement therapy or neuroactive drugs represent useful treatments for these disturbances but may be contraindicated or not accepted. Herein it was investigated the efficacy of Kava-Kava, an extract of Piper Methysticum, on mood of perimenopausal women.

Desgn: A 3-months randomized prospective open study investigating in perimenopausal women modifications induced by calcium supplementation (control; n=34), calcium plus Kava-Kava at the dose of 100 mg/day (n=15) or calcium plus Kava-Kava at the dose 200 mg/day (n=19). Anxiety was evaluated by the State Trait Anxiety Inventory (STAI); depression by the Zung's scale (SDS), and climacteric symptoms by the Greene's scale. Evaluations were performed at baseline and after 1 and 3 months.

Results: In the control group during the 3 months, anxiety, depression and climacteric symptoms tended to decline, but not significantly. During Kava-Kava anxiety declined (P<0.001) at 1 (−3.8+/−1.03) and 3 (−5.03+/−1.2) months, depression declined at 3 months (−5.03+/−1.4; P<0.002) and climacteric score declined (P<0.0006) at 1 (−2.87+/−1.5) and 3 (−5.38+/−1.3) months. Only the decline of anxiety induced by Kava-Kava was significantly greater than that spontaneously occurring in controls (P<0.009).

Conclusions: The present data indicate that, in perimenopausal women, administration of Kava-Kava induces an improvement of mood, particularly of anxiety.

Huperzine-A

Progress in studies of huperzine A, a natural cholinesterase inhibitor from Chinese herbal medicine.

Wang R, Yan H, Tang X C.

State Key Laboratory of Drug Research, Shanghai Institute of Materia Medica, Shanghai Institutes for Biological Sciences, Chinese Academy of Sciences, China.

Huperzine A (HupA), a novel alkaloid isolated from the Chinese herb Huperzia serrata, is a potent, highly specific and reversible inhibitor of acetylcholinesterase (AChE). Compared with tacrine, donepezil, and rivastigmine, HupA has better penetration through the blood-brain barrier, higher oral bioavailability, and longer duration of AChE inhibitory action. HupA has been found to improve cognitive deficits in a broad range of animal models. HupA possesses the ability to protect cells against hydrogen peroxide, beta-amyloid protein (or peptide), glutamate, ischemia and staurosporine-induced cytotoxicity and apoptosis. These protective effects are related to its ability to attenuate oxidative stress, regulate the expression of apoptotic proteins Bcl-2, Bax, P53, and caspase-3, protect mitochondria, upregulate nerve growth factor and its receptors, and interfere with amyloid precursor protein metabolism. Antagonizing effects of HupA on N-methyl-D-aspartate receptors and potassium currents may also contribute to its neuroprotection as well. Pharmacokinetic studies in rodents, canines, and healthy human volunteers indicated that HupA was absorbed rapidly, distributed widely in the body, and eliminated at a moderate rate with the property of slow and prolonged release after oral administration. Animal and clinical safety tests showed that HupA had no unexpected toxicity, particularly the dose-limiting hepatotoxicity induced by tacrine. The phase IV clinical trials in China have demonstrated that HupA significantly improved memory deficits in elderly people with benign senescent forgetfulness, and patients with Alzheimer disease and vascular dementia, with minimal peripheral cholinergic side effects and no unexpected toxicity. HupA can also be used as a protective agent against organophosphate intoxication.

Neuroprotective effects of huperzine A: new therapeutic targets for neurodegenerative disease.

Zhang H Y, Tang X C.

State Key Laboratory of Drug Research, Shanghai Institute of Materia Medica, Chinese Academy of Sciences, Zhangjiang Hi-Tech Park, Shanghai 201203, China.

In recent years, the most common pharmacological treatment for Alzheimer's disease (AD) has been acetylcholinesterase (AChE) inhibition. However, this single-target approach has limited effectiveness and there is evidence that a multitarget approach might be more effective. Huperzine A (HupA), a novel alkaloid isolated from a Chinese herb, has neuroprotective effects that go beyond the inhibition of AChE. Recent data have demonstrated that HupA can ameliorate the learning and memory deficiency in animal models and AD patients. Its potentially beneficial actions include modification of beta-amyloid peptide processing, reduction of oxidative stress, neuronal protection against apoptosis, and regulation of the expression and secretion of nerve growth factor (NGF) and NGF signaling.

The psychopharmacology of huperzine A: an alkaloid with cognitive enhancing and neuroprotective properties of interest in the treatment of Alzheimer's disease.

Zangara A.

Cognitive Drug Research, CDR House, 24 Portman Road, Reading RG30 1EA, UK. andreaz@cdr.org.uk

Huperzine A (HupA), extracted from a club moss (Huperzia serrata), is a sesquiterpene alkaloid and a powerful and reversible inhibitor of acetylcholinesterase (AChE). It has been used in China for centuries for the treatment of swelling, fever and blood disorders. It has demonstrated both memory enhancement in animal and clinical trials and neuroprotective effects. Recently it has undergone double-blind, placebo-controlled clinical trials in patients with Alzheimer's disease (AD), with significant improvements both to cognitive function and the quality of life. Most of the clinical trials are from China, but HupA and derivatives are attracting considerable interest in the West, where AD is a major and growing concern. Furthermore, both animal and human safety evaluations have demonstrated that HupA is devoid of unexpected toxicity. Other interesting aspects of HupA pharmacological profile relate to its neuroprotective properties: it has been shown in animal studies that HupA can be used as a protective agent against organophosphate (OP) intoxication and that it reduces glutamate-induced cell death.

Sleep Disorders

A sleep disorder is a condition that involves any type of difficulty that relates to sleeping. Sleep disorders are divided into two major categories. One category consists of disorders in which a person has trouble falling asleep or staying asleep. This category also includes disorders in which a person may fall asleep at inappropriate times. Conditions of these kinds are called dyssomnias. A second category of sleep disorders includes those in which people experience physical events while they are sleeping. Nightmares and sleepwalking are examples of these disorders. Conditions of this type are called parasomnias.

Dyssomnias

Insomnia: It is perhaps the most common of all sleep disorders. People with insomnia have trouble falling asleep. Often people with this disorder worry or become anxious about not being able to sleep, which can make the problem even worse. Insomnia may begin at any time in a person's life. It tends to be most common in young adulthood and middle age.

Hypersomnia: It is a condition in which a person is excessively sleepy during normal waking hours. The person may often fall asleep for lengthy periods during the day, even if he or she has had a good night's sleep. In some cases, patients have difficulty waking up in the morning. They may seem confused or angry when they awaken. The condition is most common in young adults between the ages of fifteen to thirty.

Narcolepsy: It is characterized by sleep attacks over which patients have no control. They may fall asleep suddenly with no warning. The sleep attack may last a few minutes or a few hours. The number of attacks patients experience can vary. People with narcolepsy usually feel refreshed after awakening from a sleep attack but they may become sleepy again a few hours later and experience another attack.

Sleep apnea: It is a condition in which a person actually stops breathing for ten seconds or more. The most common symptom of sleep apnea is very loud snoring. Patients with this condition alternate between periods of snoring or gasping and periods of silence.

Circadian rhythm sleep disorders: The term circadian rhythm refers to the usual cycle of activities, such as waking and sleeping that is common to any form of life. Most people are accustomed to falling asleep after it gets dark out and waking up when it gets light. In certain conditions, this pattern can be disrupted. A person may fall asleep as the sun comes up and wake up as the sun goes down. An example of a circadian sleep disorder is jet lag. People who fly suddenly across many time zones may have their sleep patterns disrupted. It may take a few days before those patterns return to normal.

Insomnia

Insomnia Causes

Insomnia may result from either psychological or physical causes.
The most common psychological problems include anxiety, stress, and depression. In fact, insomnia may be an indicator of depression. Many people will have insomnia during the acute phases of a mental illness.
Physiological causes span from circadian rhythm disorders, sleep-wake imbalance, to a variety of medical conditions. Following are the most common medical conditions that trigger insomnia:
Chronic pain syndromes
Congestive heart failure
Chronic obstructive pulmonary disease (COPD)
Degenerative diseases, such as Alzheimer disease (Often insomnia is the deciding factor for nursing home placement.)
Certain groups are at higher risk for developing insomnia:
Travelers
Shift workers
Seniors
Adolescent or young adult students
People with chronic pain, cardiopulmonary disease
Pregnant women
Women in menopause
Certain medications have been associated with insomnia. Among them are certain over-the-counter cold and asthma preparations.
The prescription varieties of these medications may also contain stimulants and thus produce similar effects on sleep.
Medications for high blood pressure have also been associated with poor sleep.
Common stimulants associated with poor sleep include caffeine and nicotine. You should consider not only restricting caffeine use in the hours immediately before bedtime but also limiting your total daily intake.
People often use alcohol to help induce sleep, as a nightcap. However, it is a poor choice. Alcohol is associated with sleep disruption and creates a sense of nonrefreshed sleep in the morning.

Insomnia Symptoms

Doctors associate a variety of signs and symptoms with insomnia. Often, the symptoms intertwine with those of other medical or mental conditions.
People with insomnia may complain of difficulty falling asleep. The problem may begin with stress. Then, as you begin to associate the bed with your inability to sleep, the problem may become chronic.
Depression and mental illnesses are often associated with insomnia.
Most often daytime symptoms will bring people to seek medical attention. Daytime problems caused by insomnia include the following:
Poor concentration and focus
Difficulty with memory
Impaired motor coordination
Irritability and impaired social interaction
Motor vehicle accidents because of fatigued, sleep-deprived drivers
People may worsen these daytime symptoms by their own attempts to treat the symptoms.
In 1995, a Gallup poll said 7.9% of respondents used alcohol to help them sleep. Alcohol and antihistamines may compound the problems with sleep deprivation.
Others have tried nonprescription sleep aids.

Effects of Insomnia
poor health and diminished quality of life
impaired social functioning
increased impatience and irritability
diminished mental alertness and memory slower reaction times and impaired concentration
increased risk of disorders such as major depression, anxiety disorder, and substance abuse
increased likelihood of automobile, home, and workplace accidents
poor job performance, missed work days, and school absences

### REFERENCES

1. Antunes F, Barclay L R C, Ingold K U. On the antioxidant activity of melatonin. Free Rad Bio Med. 1999; 26:117-128.
2. Barni S, Lissoni P, Cazzaniga M, et al. A randomized study of low-dose subcutaneous interleukin-2 plus melatonin versus supportive care alone in metastatic colorectal cancer patients progressing under 5-fluorouracil and folates. Oncology. 1995; 52:243-245.
3. Brzezinski A. Melatonin in humans. N Engl J. Med. 1997; 336:186-195.
4. Bursztajn H J. Melatonin therapy: from benzodiazepine-dependent insomnia to authenticity and autonomy. Arch Intern Med. 1999; 159:2393-2395.
5. Cupp M J. Melatonin. Am Fam Physician. 1997; 56:1421-1425.
6. Dolberg O T, Hirschmann S, Grunhaus L. Melatonin for the treatment of sleep disturbances in major depressive disorder. Am J. Psychiat. 1998; 155:1119-1121.
7. Force R W, Hansen L, Bedell M. Psychotic episode after melatonin [letter]. Ann Pharmacother. 1997; 31:1408.
8. Garfinkel D, Zisapel N, Wainstein J, Laudon M. Facilitation of benzodiazepine discontinuation by melatonin. Arch Intern Med. 1999; 159:2456-2460.
9. Hartter S, Grozinger M, Weigmann H, et al. Increased bioavailability of oral melatonin after fluvoxamine administration. Clin Pharmacol Therap. 2000; 67:1-6.
10. Middleton B A, Stone B M, Arendt J. Melatonin and fragmented sleep patterns. Lancet. 1996; 348:551-552.
11. Murphy P J, Myers B L, Badia P. Nonsteroidal anti-inflammatory drugs alter body temperature and suppress melatonin in humans. Physiol Behav. 1996; 59:133-139.
12. Reiter R J. Melatonin, active oxygen species and neurological damage. Drug News Perspect. 1998; 11:291-296.
13. Reppert S M, Weaver D R. Melatonin madness. Cell. 1995; 83:1059-1062.
14. Sainz R M, Mayo J C, Reiter R J, et al. Melatonin regulates glucocorticoid receptor: an answer to its antiapoptotic action in thymus. FASEB J. 1999; 13:1547-1556.
15. Turjanski A G, Rosenstein R E, Estrin D A. Reactions of melatonin and related indoles with free radicals: a computational study. J Med. Chem. 1998; 44:3684-3689.
16. Voorduow B C, Euser R, Verdonk R E, et al. Melatonin and melatonin-progestin combinations alter pituitary-ovarian function in women and can inhibit ovulation. J Clin Encocrinol Metab. 1992; 74:108-117.
17. Wiid 1, Hoal-van Helden E, Hon D. et al. Potentiation of isoniazid activity against Mycobacterium tuberculosis by melatonin. Antimicrob Agents Chemother. 1999; 43:975-977.
18. Cheng D H, Tang X C. Comparative studies of huperzine A, E-2020 and tacrine on behavior and cholinesterase activities. Pharmacol Biochem Behav. 1998; 60:377-386.
19. Cheng D H, Ren H, Tang X C. Huperzine A, a novel promising acetylcholinesterase inhibitor. Neuroreport. 1996; 8:97-101.
20. Quian B C, Wang M, Zhou Z F, et al. Pharmacokinetics of tablet huperzine A in six volunteers. Chung Kuo Yao Li Hsuch Pao. 1995; 16:396-398.
21. Tang X C, Kindel G H, Kozikowski A P, Hanin I. Comparison of the effects of natural and synthetic huperzine A on rat brain cholinergic function in vitro and in vivo. J. Ethnopharmacol. 19
22. Xiong Z Q, Tang X C. Effect of huperzine A, a novel acetylcholinesterase inhibitor, on radial maze performance in rats. Pharmacol Biochem Behav. 1995; 51:415-419.
23. Xu S S, Gao Z X, Weng Z, et al. Efficacy of tablet huperzine-A on memory, cognition and behavior in Alzheimer's disease. Chung Kuo Yao Li Hsuch Pao. 1995; 16:391-395.
24. Ye J W, Cai J X, Wang L M, Tang X C. Improving effects of huperzine A on spatial working memory in aged monkeys and young adult monkeys with experimental cognitive impairment. J Pharmacol Exp Ther. 1999; 288:814-819.
25. Zhang R W, Tang X C, Han Y Y, et al. Drug evaluation of huperzine A in the treatment of senile memory disorders. [Article in Chinese] Chung Kuo Yao Li Hsuch Pao. 1991; 12:250-252.

We claim:

1. A sleep inducing composition comprised of the following ingredients:
a. 33.33% Kava-Kava
b. 33.33% Melatonin
c. 33.33% Huperzine-A which is designed to promote deep and restful sleep.

2. A strip form delivery system composition of claim 1 comprised of the following ingredients
a. 33.33% Kava-Kava
b. 33.33% Melatonin
c. 33.33% Huperzine-A

3. Increase Sleep
a. Fast Asleep strips help to induce a state of relaxation and relieve tension by:
b. administered by strip delivery system with an effective amount of composition of claim 1.
c. administered by strip delivery system with an effective amount of composition of claim 2.

4. Treat insomnia.
a. Fast Asleep strips help to improve the ability to fall asleep and stay asleep.
b. administered by strip delivery system with an effective amount of composition of claim 1.
c. administered by strip delivery system with an effective amount of composition of claim 2.

5. Relieve Stress and Anxiety
a. Fast Asleep strips are non-sedating supplements, which promote relaxation, calmness and a feeling of well being.
b. administered by strip delivery system with an effective amount of composition of claim 1.
c. administered by strip delivery system with an effective amount of composition of claim 2.

6. Boost Immune System
a. Ingredients in Fast Asleep Strips have the ability to act counter immune-supressing effects of Cotisol
b. Act as an antioxidant which aids in preventing and reducing the damage done to the body by free radicals.
c. administered by strip delivery system with an effective amount of composition of claim 1.
d. administered by strip delivery system with an effective amount of composition of claim 2.

7. Increase Life Span
a. Melatonin, a key ingredient in Fast Asleep, slows the ageing process by acting as an antioxidant:
b. Melatonin is the most active and effective of all naturally occurring antioxidant compounds and is capable of penetrating the cells of the whole body.
c. administered by strip delivery system with an effective amount of composition of claim 1.
d. administered by strip delivery system with an effective amount of composition of claim 2.

8. Improve Mental Clarity
a. Fast Asleep contains ingredients which have been shown to improve concentration, memory and reaction time for people suffering from anxiety.by:
b. administering an effective amount of composition of claim 1.
c. administering an effective amount of composition of claim 2.

9. Enhance Memory
a. Fast Asleep contains Huperzine A an alkaloid found in the Chinese herb Huperzia serrata.
b. Huperzia A is used to improve memory, focus and concentration.
c. Huperzia A helps alleviate memory problems amount the elderly.
d. Huperzia A could be a treatment for diseases characterized by neurodegeneration-particularly Alzheimer's Disease.
e. administered by strip delivery system with an effective amount of composition of claim 1.
f. administered by strip delivery system with an effective amount of composition of claim 2.

10. Pain Reliever
a. The botanical, Kava, a key ingredient in Fast Asleep, has been shown to be an excellent analgesic and muscle relaxant.
b. Kava is more effective than aspirin, but not as potent as morphine
c. administered by strip delivery system with an effective amount of composition of claim 1.
d. administered by strip delivery system with an effective amount of composition of claim 2.
